(19) United States
(12) Patent Application Publication
(10) Pub. No.: US 2008/0318332 A1
(43) Pub. Date: Dec. 25, 2008

(54) DISEASE DIAGNOSIS BY PROFILING SERUM GLYCANS

(76) Inventors: Yehia S. MECHREF, Bloomington, IN (US); Milos V. NOVOTNY, Bloomington, IN (US); Zuzana KYSELOVA, Povazska (SK); Pilsoo KANG, Bloomington, IN (US)

Correspondence Address: BARNES & THORNBURG LLP, 11 SOUTH MERIDIAN, INDIANAPOLIS, IN, 46204, US

(21) Appl. No.: 12/127,366

(22) Filed: May 27, 2008

Related U.S. Application Data
(60) Provisional application No. 60/940,521, filed on May 29, 2007.

(51) Int. Cl. G01N 33/66

(52) U.S. Cl. 436/106

(57) ABSTRACT

Specific glycomic profiles derived from human blood sera of breast cancer patients and prostate cancer patients were compared to those of disease-free females and males, respectively. The profiles were acquired using MALDI-MS of permethylated N-glycans released from 10-μl sample aliquots. Quantitative permethylation was attained using solid-phase permethylation. Principal component analysis of the glycomic profiles revealed significant differences among the two sets (diseased vs. non-diseased), allowing their distinct clustering. Several sialylated and fucosylated N-glycan structures were found to be useful as biomarkers for cancer, particularly breast cancer or prostate cancer.

### TECHNICAL FIELD

The present disclosure pertains to profiling glycomic changes in sera using mass spectrometry.

### BACKGROUND

Glycosylated proteins play important roles in cell-to-cell interactions, immunosurveillance, and a variety of receptor-mediated and specific protein functions through a highly complex repertoire of glycan structures. Correspondingly, metabolic dysfunctions and disease states may be reflected in appearance of abnormal glycans and/or altered quantitative proportions within the glycome. Such observations provide a rationale for the recent developments in the field of functional glycomics. Differential glycomic measurements between healthy and disease states may have clinical diagnostic potential, as exemplified by the recent utilization of capillary electrophoresis in diagnosing liver cirrhosis.

Certain links between cancer diseases and altered protein glycosylation have been noted for both N-linked and O-linked glycoconjugates, with knowledge of abnormal glycosylation in this set of diseases coming from the investigations of tumor tissues and cell cultures. Detecting cancer-related changes in patients' blood has been a distinct goal of recent studies, but this has been less commonly pursued due to methodological difficulties with high-sensitivity measurements of glycans. Coincidentally, some recently identified cancer biomarkers in human serum are glycoproteins, usually the large, mucin-type molecules.

Aberrant glycosylation has been implicated in various types of cancer, where many glycosyl epitopes constitute tumor-associated antigens. Molecular changes in glycosylation may be associated with the signaling pathways of the malignant transformation of cells. There is a correlation between certain structures of glycans and a clinical prognosis in cancer. Structural studies of glycans and other related molecules on cellular surfaces have been performed. Due to the limited specificity of cancer diagnostic tests currently available, it is desirable to develop more diagnostically informative procedures for blood analysis in cancer cases.

### SUMMARY OF THE INVENTION

A method for monitoring glycomic change in a biological system in accordance with the present invention comprises one or more of the following features or combinations thereof:

The profiles of N-glycans released from glycoproteins of human serum are shown to be indicative of the different stages of breast cancer. While using small (10 μL) serum aliquots, we have been able to perform sensitive and quantitative mass spectrometric (MS) measurements of the constituent profiles of N-glycans originating from circulating proteins. These profiles were further evaluated statistically through pattern recognition techniques (principal component analysis, PCA) in terms of breast cancer disease stage. The obtained N-glycan data cluster well for different stages, which further differ from a data set recorded from individuals apparently free of the disease. The clusters readily distribute into groups that correlate with the stage of breast disease determined by pathological assessment. This type of clinically useful information was obtained from a small volume of blood serum, without biopsy or tumor removal. To implicate certain oligosaccharide structures as biomarkers, we have also carried out additional statistical evaluations (data mining) through non-parametric Receiver Operating Characteristic (ROC) and ANOVA analyses of approximately 50 individual N-glycans. For comparison, we have also examined the glycomic profiles of breast cancer cell lines, both invasive and non-invasive types, while confirming that they resemble the glycan patterns derived from patient specimens.

A methodological approach involving MS measurements has been devised in which a small volumetric aliquot (10 μl) of serum provides sufficient amounts of sample to display the N-linked glycan structures in a profile (or a “glycomic map”) that is statistically indicative of the presence of cancer, such as prostate cancer or breast cancer. The study has involved recording and comparing the glycomic profiles from the blood sera of healthy individuals and prostate cancer patients. This procedure deals with the sum, or a “glycan reservoir,” of protein-free molecular entities whose total profile (qualitatively or quantitatively) becomes diagnostically indicative. Further, the methodology has been used to identify twelve (12) biomarker candidates for cirrhosis and liver cancer.

In the procedures described herein, the quantitative nature of MS profiling and its sensitivity have been strengthened by the application of a permethylation procedure which allows a simultaneous quantitative recording of both neutral and acidic glycans. The overall procedure is sensitive, relatively rapid, and reliable.

### BRIEF DESCRIPTION OF THE DRAWINGS

FIG. 1 shows MALDI mirror mass spectra of permethylated N-glycans derived from a 10-μl aliquot of the serum of (A) healthy individual (upper trace) and stage I breast cancer patient (lower trace) and (B) a healthy individual (upper trace) and stage IV breast cancer patient (lower trace). Symbols: filled square, N-acetylglucosamine; grey circle, mannose; open circle, galactose; filled triangle, fucose; and filled diamond, N-acetylneuraminic acid;

FIG. 2 shows Principal Component Analysis (PCA) score plots for MALDI/MS of N-glycans derived from the sera of healthy individuals and breast cancer patients at different stages of the disease;

FIG. 3 shows representative ROC analysis AUC plots for different N-glycan structures reflecting high-(A), moderate-(B), and low-accuracy (C) tests;

FIG. 4 shows relative intensity changes of 8 N-glycans with high accuracy ROC analysis AUC values (0.9<AUC<1 or 0<AUC<0.1) and ANOVA test p-values less than 0.001 for healthy vs. breast cancer stages;

FIG. 5 shows relative intensities of N-glycans derived from membrane bound proteins isolated from normal (MCF10A), invasive (MDA-MB-231, MDA-MB-435), and non-invasive (578T, ADR-RES, BT549 and T47D) cancer cells;

FIG. 6 shows bar graphs of N-glycan relative intensity changes derived from the blood sera of healthy individuals vs. breast cancer patients according to their structural type;

FIG. 7 shows MALDI mirror spectra of permethylated N-glycans derived from human blood serum of a healthy individual vs. a prostate cancer patient. Symbols: filled square, N-acetylglucosamine; filled circle, mannose; open circle, galactose; filled triangle, fucose; and filled diamond, N-acetylneuraminic acid;

FIG. 8 shows Principal Component Analysis (PCA) score plots for MALDI/MS of N-glycans derived from blood sera of healthy individuals (n=1 0) and prostate cancer patients (n=24);

FIG. 9 shows representative ROC analysis AUC plots for different glycan structures reflecting high-(a), moderate-(b), and low-accuracy (c) tests;

FIG. 10 shows MALDI mass spectra representative of the N-glycans derived from human blood serum of a healthy individual and a prostate cancer patient;

FIG. 11 shows box graphs comparing the average relative intensities of all N-glycans which demonstrated highly accurate ROC analysis AUC values (0.9<AUC<1; 0<AUC<0.1) and ANOVA test p-values lower than 0.001, for 10 healthy and 24 prostate cancer samples;

FIG. 12 shows Principal Component Analysis (PCA) score plots for MALDI/MS of N-glycans derived from the sera of cancer-free individuals, liver cancer patients, and cirrhosis patients;

FIG. 13 shows box graphs comparing the average relative intensities of all N-glycans which demonstrated highly accurate ROC analysis AUC values (0.9<AUC<1; 0<AUC<0.1) and ANOVA test p-values lower than 0.001, for 77 cancer-free, 73 liver cancer, and 53 cirrhosis samples; and

FIG. 14 shows ANOVA p-values for 12 biomarker candidates for cirrhosis and liver cancer. Symbols: filled square, N-acetylglucosamine; grey circle, mannose; open circle, galactose; and filled diamond, N-acetylneuraminic acid.

### RESULTS AND DISCUSSION

While the invention is susceptible to various modifications and alternative forms, specific embodiments will herein be described in detail. It should be understood, however, that there is no intent to limit the invention to the particular forms described, but on the contrary, the intention is to cover all modifications, equivalents, and alternatives falling within the spirit and scope of the invention.

The association of cancerous cells with unusual glycosylation of their surface proteins, and their subsequent shedding into the circulating fluids, provides an opportunity for diagnosing diseases, such as cancer. In order to recognize reliably the relevant glycan structures which may be associated with pathologically distinct glycoconjugates, it is advantageous to display many candidate glycans in a compound profile at sufficiently high sensitivity. Human blood is a rich source of structurally and functionally diverse glycoproteins, their numerous glycosylation sites and complex microheterogeneities necessitate highly sensitive techniques. Modern biomolecular mass spectrometry (MS) supports the detection, separation, identification, and display of multiple glycans in glycomic investigations.

### Breast Cancer

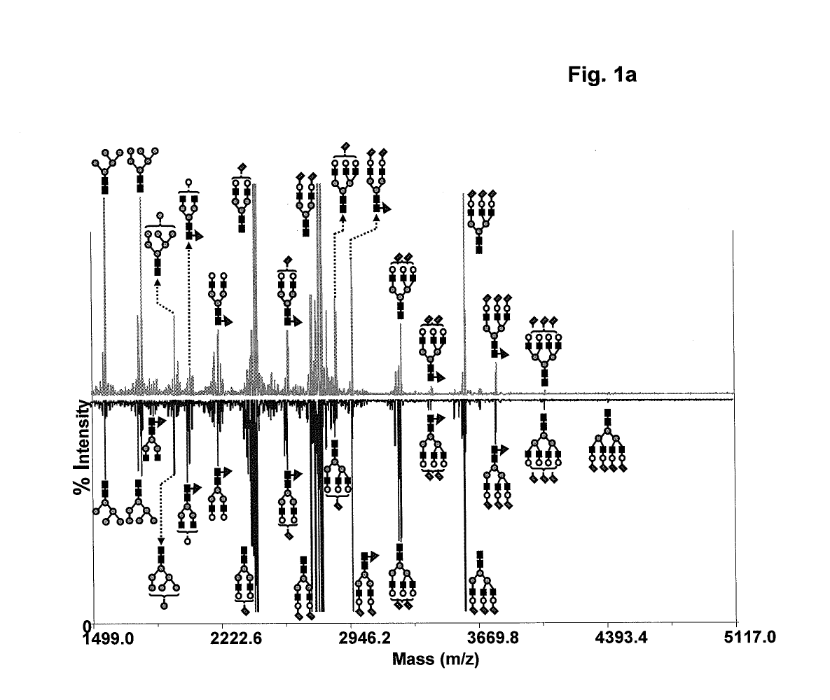

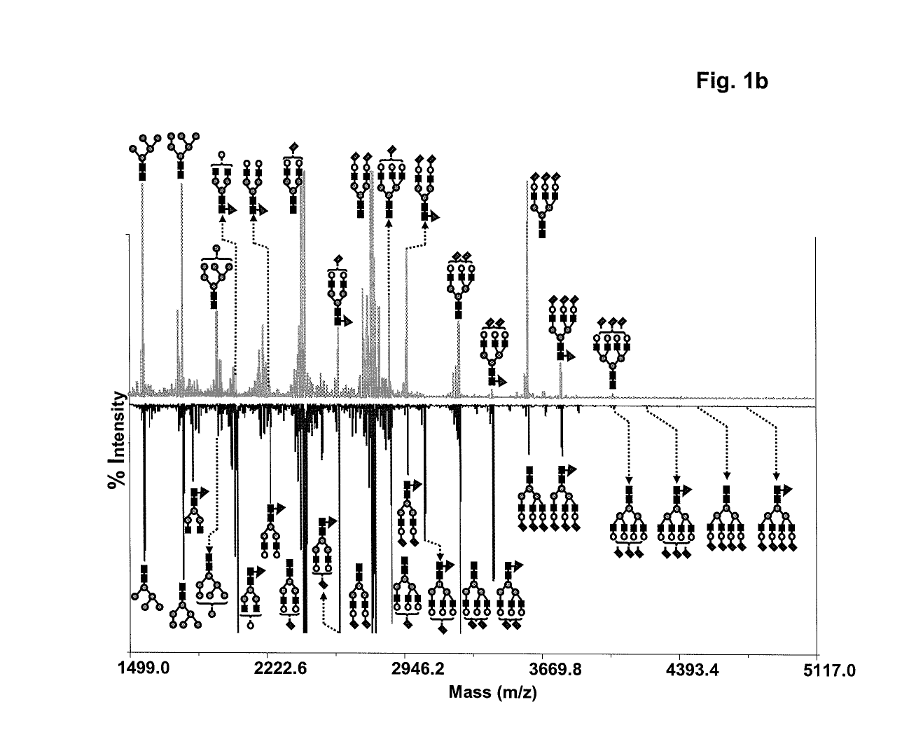

Serum Glycomic Profiles. In total, we identified and quantified approximately 50 different N-glycan structures, which cover typical structural types, including high-mannose, hybrid and complex-type entities. The high-energy collision process used in this type of tandem mass spectrometry (MS/MS) yields reliable structural identification of each recorded glycan. Tandem mass spectrometry was employed to identify the majority of the structures. In comparing visually the glycomic profiles of the healthy individuals with those suffering from breast cancer, we found that, within the molecular mass range of 1,500 to 5,000 m/z, there was an overall decrease in abundance of smaller N-glycans (m/z: 1,500-2,700) at the expense of larger molecules. Representative profiles are illustrated in FIG. 1a and 1b, with a healthy profile compared to stage I and IV, respectively. These relative ion intensity changes in a profile (i.e., small N-glycans changing into larger structures due to differential addition of sugar residues) were enhanced from stage I to stage IV.

Principal Component Analysis (PCA) of N-Glycan Profiles. In assessing more precisely the patterns of N-glycans that could be characteristic for specific cancer conditions, we turned to PCA which is designed to capture variance in a data set. PCA involves a set of variables that define a projection encapsulating a data set, which is orthogonal (uncorrelated) to the previous principal component.

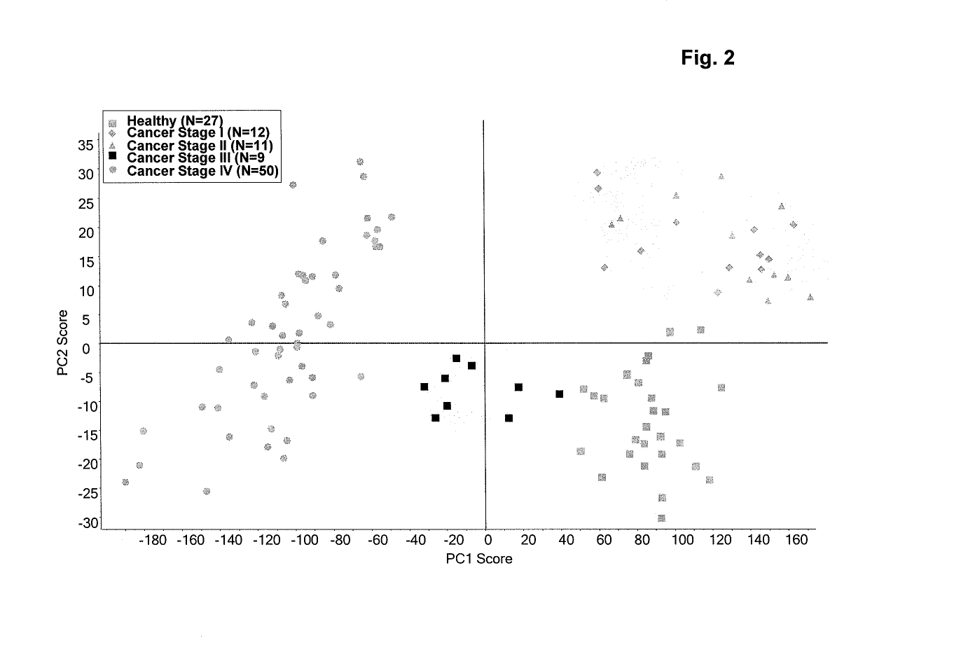

The acquired profile data, measured for all subjects (both healthy and breast cancer patients) were subjected to PCA as described above (FIG. 2). The number of subjects in the early stages of breast cancer (stage I, n=12; stage II, n=11; stage III, n=9) that were available was lower than for stage IV (n=50) and individuals free of the disease (n=27). Through the process of acquiring and evaluating these data in time, we obtained reliable and discrete distribution of spots. The final clustering of our data indicated significant differences in the glycomic profiles of healthy individuals in comparison with the early stages of breast cancer. This observation supports a diagnostic potential for recognizing different stages of breast cancer and possible detection of early onset through glycomic analysis of serum specimens.

Glycan Biomarker Search Through Data Mining. A further statistical evaluation of putative biomarkers was performed through non-parametric Receiver Operating Characteristic (ROC) procedure. ROC is commonly used in the test situations where diagnostic indicators yield numerical results that can be compared to an independent diagnosis, confirming either the presence or absence of a disease. The ROC analysis was achieved using AccuROC 2.5 software for Windows (Accumetric Corporation, Montreal, Canada) which was designed for medical applications, operating in two parameters: sensitivity and specificity. It evaluates the area under the (ROC) curve (AUC) and uses this value to describe performance of the overall cutpoints.

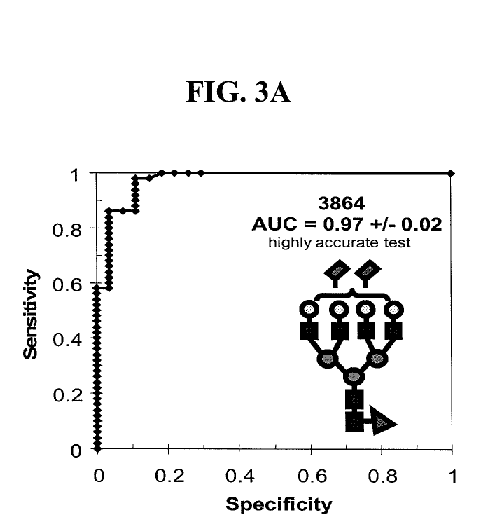

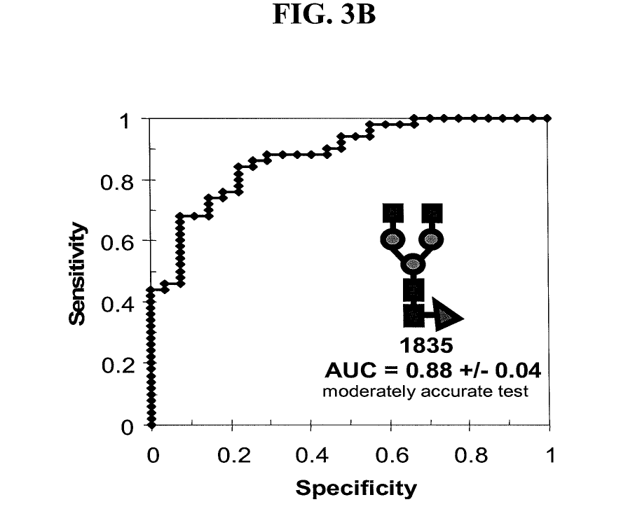

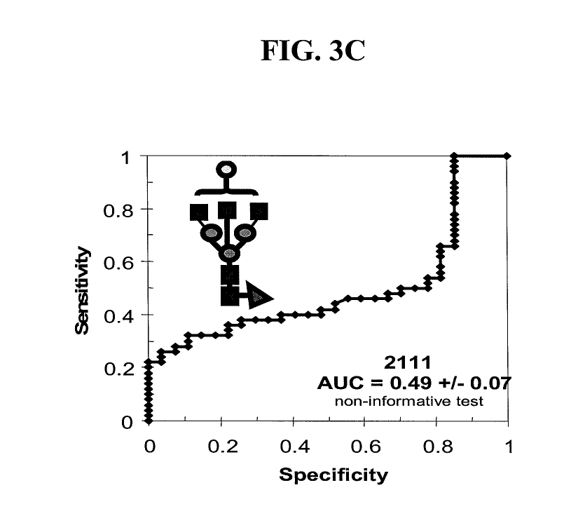

FIG. 3 shows some examples of N-glycan AUC outputs in the ROC test, comparing healthy individuals and stage IV breast cancer patients. The N-glycan with m/z of 3,864 has AUC value of 0.97, yielding a highly accurate potential biomarker indicator (FIG. 3A), while another structure (m/z=2,111) has an AUC value of only 0.49, classifying it as a non-informative (unlikely) correlation (FIG. 3C). An AUC-value of 0.88 was calculated for the N-glycan with m/z of 1,835, making it only a moderately accurate test (FIG. 3B). When comparing the expression of specific N-glycan structures in healthy individuals with the expression of the same structure in breast cancer patients as a function of their disease status, the AUC-values reflect the trend for a particular N-glycan. If AUCs are over the 0.5 cut-off point and approaching 1, it means not only increasing the value of the observed diagnostic parameter but also improving the accuracy and reliability of the used test. However, in several cases, the AUC-values dropped below 0.5 limit, indicating the opposite trend: the corresponding relative intensity changes in N-glycans were decreasing in the direction of disease progression. Generally, the further away from AUC=0.5 the actual N-glycan values are found; the broader the observed glycosylation changes, with a correspondingly higher accuracy of the ROC analysis.

Table 1 lists the N-glycans that were evaluated in terms of their AUC-values as well as their p-values from yet another statistical procedure: Single Factor Analysis of Variance (ANOVA) test. We have taken into consideration only the N-glycan cases with the comparison of non-diseased and diseased experimental groups which yielded the p-values <0.05. In this table, the light grey highlighted rows are the N-glycans whose AUC-values place them in the high-accuracy interval (0.9<AUC<1 or 0<AUC<0.1). The dark grey highlighted rows represent those in the moderately accurate interval (0.7<AUC<0.9 or 0.1<AUC<0.3), while the white rows belong to the structures with AUC-values of less accuracy (0.5<AUC<0.7 or 0.3<AUC<0.5).

TABLE 1
The ROC analysis AUC values from and from ANOVA test p-values of N-glycans derived from the serum of healthy
individuals and patients exhibiting stage I-IV breast cancer.

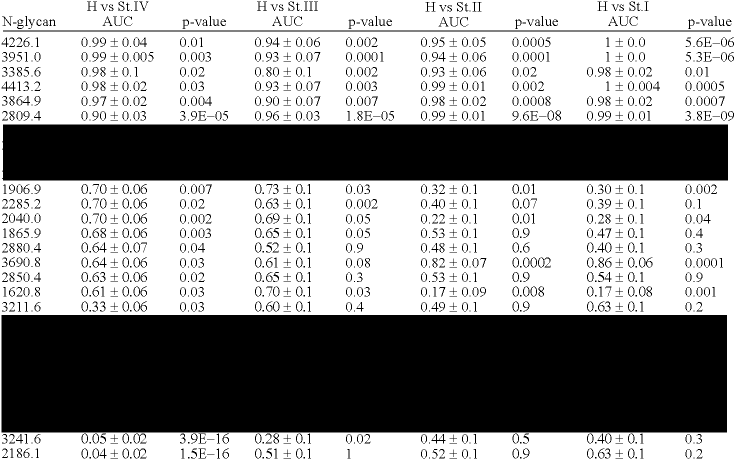

Light grey highlighted rows are the N-glycans with ROC analysis AUC values reflecting their high accuracy (0.9 < AUC < 1 or 0 < AUC < 0.1) as tested by ROC. Dark grey highlighted rows are the N-glycans with ROC analysis AUC values reflecting their moderate accuracy (0.7 < AUC < 0.9 > or 0.1 < AUC < 0.3), while the white rows represent the N-glycans with ROC analysis AUC values reflecting their low accuracy (0.5 < AUC < 0.7 or 0.3 < AUC < 0.5).

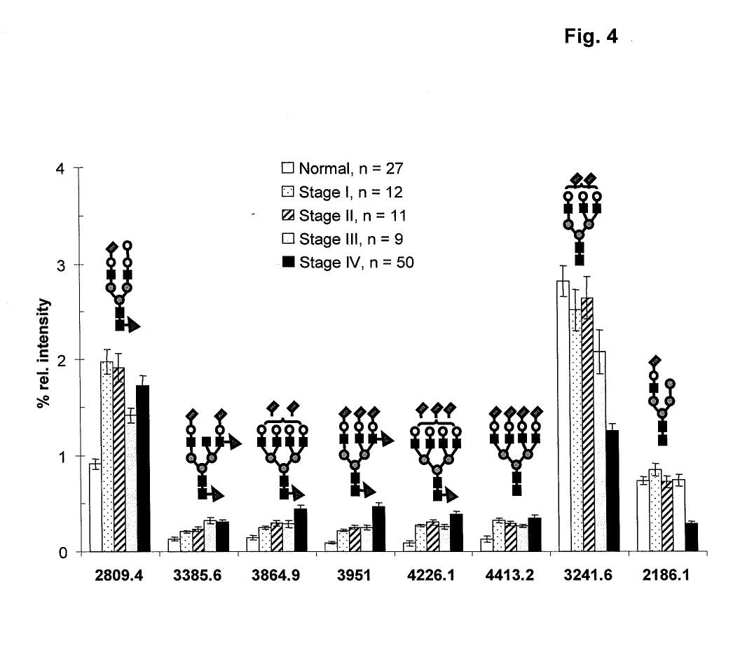

A further correlation of the favorable AUC-values with p-values from ANOVA, has implicated eight N-glycans whose relative intensity changes profoundly during breast cancer development (FIG. 4). These particular N-glycans thus represent strong biomarker candidates, as the occurrence of their specific structures has been confirmed by two independent statistical approaches, both accepted widely in the biomedical literature.

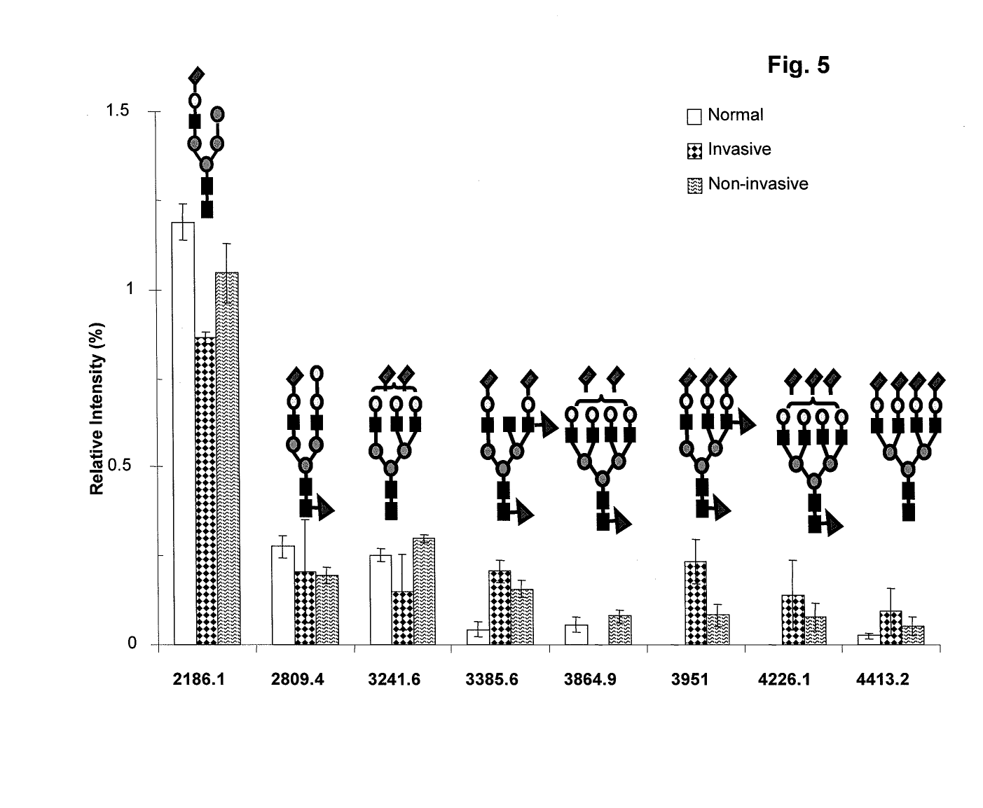

We supplemented our investigations on serum glycan levels by the parallel analyses of a human mammary epithelium cell (control) and cancer cell lines (invasive and non-invasive type) through the same analytical procedure. While examining the eight N-glycan structures implicated in the blood serum as pertinent to cancer, we found similar trends for six of these structures in the extracts prepared from invasive (MDA-MB-231, MDA-MB-435) and noninvasive (578T, ADR-RES, BT549 and T47D) breast cancer cells (see FIG. 5). The invasive cell lines, capable of forming tumors away from their original site of transformation, were found to result in a more dramatic change in the glycomic pattern when compared to the non-invasive cells. Moreover, N-glycans associated with m/z 3951 and 4226 are not observed in normal cell lines, suggesting their potential role during tumorigenesis process.

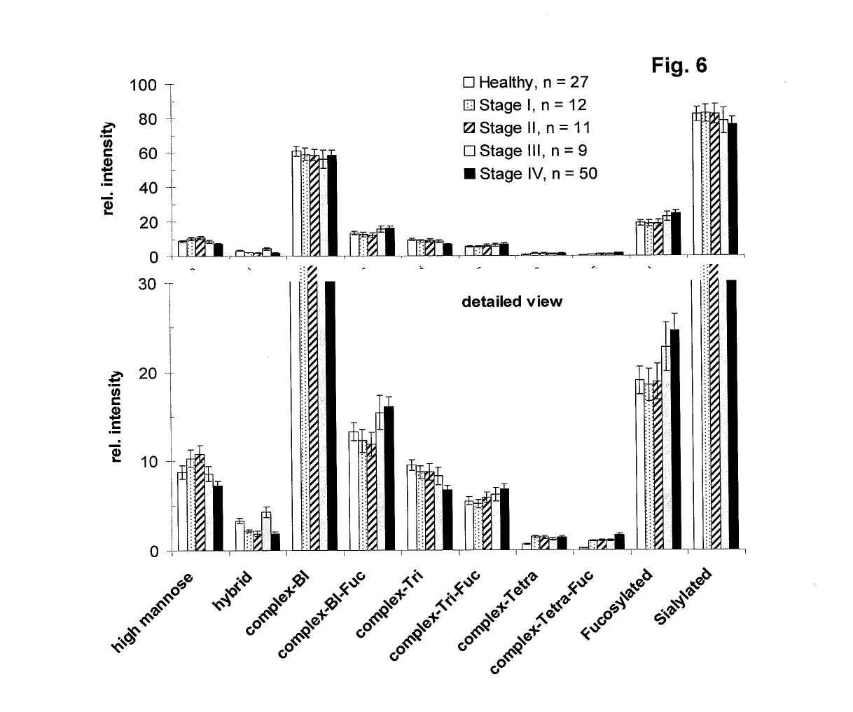

Structural Correlations. Grouping N-glycan profile constituents into categories according to molecular size, number of antennas and sugar residue abundance (FIG. 6) can be indicative of certain general trends due to a disease progression. The overall trend in increasing the N-glycan size due to the addition of sialic acid residues and enhanced fucosylation is evident. All eight N-glycans selected by AUC-values and ANOVA are sialylated to a different degree (mono-, di-, tri- and tetra-sialylated). Moreover, five out of these structures are fucosylated (two of them di-fucosylated), supporting the general notion of fucosylation involvement during progression of cancer in a different organ.

Discussion. Additional validation for clinically relevant glycomic mapping with serum comes from the correspondence of implicated N-glycan structures with those found in cancer cell lines in association with membrane glycoproteins. As shown in FIG. 4 and FIG. 5, and verified through statistical criteria, there is a more intense glycosylation associated with cancerous cells. In our measurements, the invasive cell lines capable of forming tumors away from their original site of transformation, represented a more extreme change in glycomic pattern when compared to the non-invasive cells.

Six N-glycan structures from the cancer cell lines are shared with those implicated as significant in our serum measurements (eight major N-glycans). The corresponding differences in glycomic maps revealed an increase of fucosylation (both within the core component and the branched segments) with malignant transformation. Increased fucosylation has been associated also with pancreatic cancer, colorectal cancer, human leucocyte cancer and renal carcinomas. A less clear situation emerges with the serum measurements of various sialylated structures in serum. Various sialylated oligosaccharides are implicated in different malignant cells as both O-linked and N-linked structures.

In conclusion, the MS-based glycomic profiling of serum-derived constituents described herein provides a highly sensitive and informative approach to differential evaluation of cancer conditions based on the aberrant glycobiology of cancerous cells and cells shedding their surface glycoproteins into the surrounding biofluids. While it is not yet clear which serum glycoproteins are primarily responsible for the diagnostically distinct N-glycans, statistical analyses of selected N-glycans (or their patterns) can be used for developing diagnostic/prognostic procedures. The method described herein provides nearly absolute structural information at a moderate level of measurement throughput. The PCA, ROC and ANOVA statistical analyses from these measurements have independently confirmed eight N-glycans with >95% of relevance to breast cancer (p<0.001), while additional glycan structures still might contribute to distinction in the recognized patterns (different stages). The glycan profiling analyses consume only minute volumes of serum sample aliquots representing essentially a non-invasive methodology for diagnosis.

### Prostate Cancer

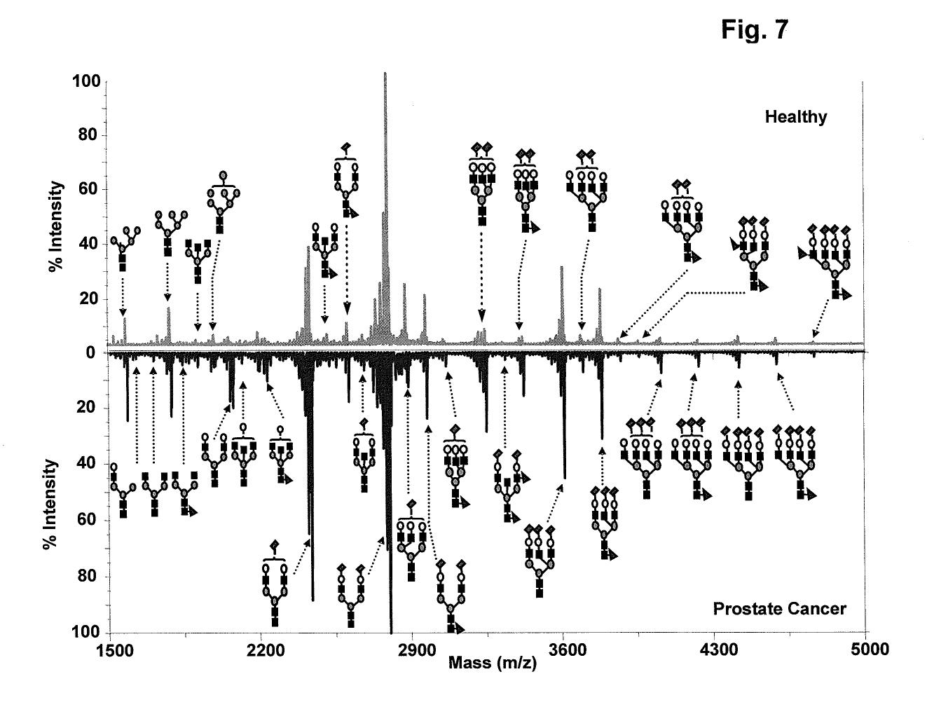

Glycomic Profiles Derived From Human Sera. The comparative glycomic approach utilized here allowed quantitative distinction between the glycan structures derived from the sera of healthy individuals and metastatic prostate cancer patients. The profiles of permethylated N-glycans derived from 10 μl of serum volumes were recorded for the m/z range of 1500-5000 using MALDI-MS. The profiles generally appeared as different between the two sample sets. Representative mirror spectra for the N-glycans derived from a healthy individual and a prostate cancer patient are seen in FIG. 7. Structural assignment of the different glycans depicted in FIG. 7 were based on both enzymatic sequencing using exoglycosidases and tandem MS.

Principal Component Analysis (PCA) of Measured Spectra. For an informative statistical analysis of the acquired glycomic profiles, the PCA procedure was employed, as is commonly used in microarray research for cluster analysis. PCA is designed to capture a variance in the given data sets in terms of their principal components, meaning a set of variables which defines a projection encapsulating the maximum amount of variation in a dataset. It is orthogonal (and, therefore, uncorrelated) to the previous principal component. PCA is a chemometric tool which is commonly employed to establish the differences among sample sets.

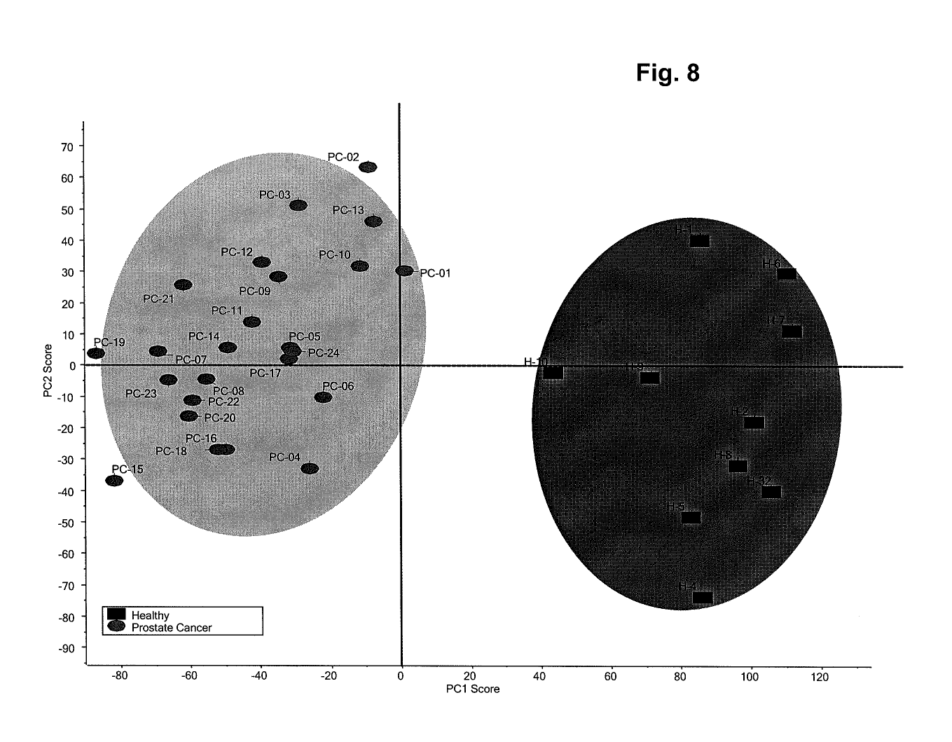

A plot of the scores of principal component one and two for the healthy and prostate cancer samples is illustrated in FIG. 8. The two sets of samples received distinguishable first principal component (PC1) scores. Thus, the healthy samples received positive PC1 scores, while the cancer samples attained negative PC1 scores. Consequently, the two sets clustered in a manner allowing the distinction between the glycomic profiles derived from healthy individuals and those derived from prostate cancer patients (FIG. 8). This shows that glycomic profiling can be used as a diagnostic tool to discriminate between diseased and non-diseased states, with the potential to detect an early stage of cancer.

Changes in Intensities for Particular Structures. Classification of N-glycans into structural groups could be another means to determine differences in the glycosylation patterns. The relative intensities of different N-glycans detected in both healthy and prostate cancer samples were compared according to the glycan types, including classification into high-mannose, complex biantennary (with or without fucosylation), complex triantennary (with or without fucosylation), complex tetraantennary (with or without fucosylation), and, hybrid, fucosylated and sialylated types. The results of the comparison are shown in Table 2.

TABLE 2
Changes in the relative intensities of the types of N-glycans derived from
healthy individuals and prostate cancer patients.

| | Healthy | | |
|---|---|---|---|
| | Individuals, | Prostate Cancer | |
| | n = 10 | Patients, n = 24 | |
| | Relative intensity | Relative intensity | ANOVA |
| Glycan Type | (%) ± SEM | (%) ± SEM | p-values |
| High mannose | 16.96 ± 0.65 | 13.45 ± 0.79 | 0.007 |
| Hybrid | 2.94 ± 0.25 | 3.13 ± 0.17 | 0.5^{NS} |
| Complex - Bi | 39.80 ± 1.65 | 35.54 ± 0.97 | 0.0005 |
| Complex - Bi - Fuc | 13.34 ± 1.22 | 13.42 ± 0.65 | 0.1^{NS} |
| Complex - Tri | 13.92 ± 0.98 | 16.10 ± 0.78 | 0.01 |
| Complex - Tri - Fuc | 10.36 ± 0.60 | 13.76 ± 0.90 | 0.002 |
| Complex - Tetra | 1.88 ± 0.18 | 2.74 ± 0.18 | 0.005 |
| Complex - Tetra - Fuc | 1.25 ± 0.11 | 1.98 ± 0.20 | 0.007 |
| Fucosylated | 24.96 ± 1.93 | 29.16 ± 1.75 | 0.0006 |
| Sialylated | 77.36 ± 4.48 | 78.54 ± 3.37 | 0.2^{NS} |

^{NS}not significant

A decrease in the relative intensities of high-mannose and complex biantennary structures and the concomitant increase in the fucosylated complex biantennary, complex tri- and tetraantennary N-glycans (both fucosylated and non-fucosylated) was consistent among all samples, exhibiting the overall ANOVA test p-values lower than 0.007. An overall decrease in smaller N-glycans (m/z 1500-2700) was also observed for the cancer samples, consistent with an overall increase of the larger N-glycans (m/z 2700-5000). The alteration in glycosylation has been correlated to tumor progression. The differences observed in our work among the high-mannose, complex biantennary, complex triantennary (fucosylated and non-fucosylated), and complex tretra-antennary (fucosylated and non-fucosylated) show ANOVA test p-values better than 0.01, indicating there is a less than 1% probability that this difference is due to a chance alone. An increase in the overall fucosylation in the case of the cancer samples is statistically significant, as suggested by the ANOVA test p-value of 0.0006.

ROC and ANOVA Analyses. A statistical evaluation of changes in the intensities associated with all N-glycans that were observed in the MALDI mass spectrum was further performed to validate the aforementioned differences for the individual glycans. Such statistical evaluations were performed using two independent approaches: ANOVA and ROC curve analyses. ROC analysis is used in the test situations where the diagnostic test yields numerical results that can be compared to an independent diagnosis, confirming either the presence or absence of a disease. The software was designed for a medical use and operates on two parameters: sensitivity and specificity. The AccuROC software evaluates the area under the ROC curve (AUC), which numerically describes performance of a particular analysis. AUC is a combined measure of sensitivity and specificity and thus the overall performance of a diagnostic test, which can be interpreted as the average value of sensitivity for all possible values of specificity. It can take on any value between 0 and 1, since both x and y axes can have values in that range. The closer AUC is to 1, the better the overall diagnostic performance of this test, while a test with an AUC value of 1 is the one that is perfectly accurate. A test is considered to be highly accurate for the AUC values of 0.9 or higher, while a moderately accurate test demonstrates an AUC value between 0.7-0.89. A test with an AUC lower than 0.7 is considered to be inaccurate.

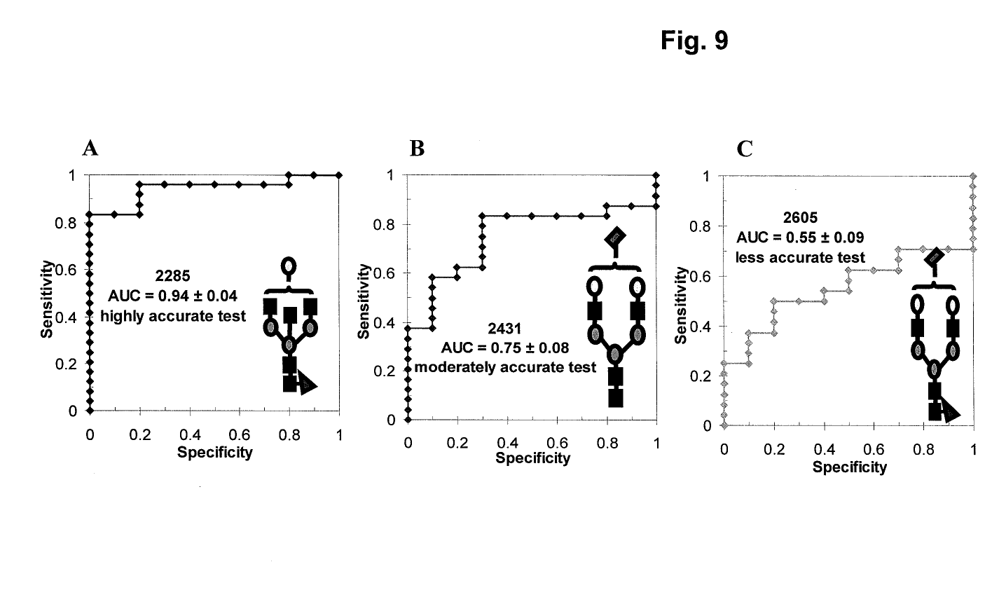

Some illustrative examples of the ROC curves for different N-glycans are shown in FIG. 9, while using the three criteria of evaluation that are commonly observed with ROC test AUC values. The N-glycan with an m/z value of 2285 demonstrates an AUC value of 0.94±0.04 (FIG. 9a), thus making this particular structure highly predictive of the disease state. The AUC value of m/z 2431 is 0.75±0.08 (FIG. 9b), suggesting a moderate accuracy. An AUC value of 0.55±0.09, calculated for the N-glycan with mass of 2605 is suggesting a lack of cancer specificity for this structure (FIG. 9c). Generally, the AUC values are believed to reflect the trends in the relative intensity changes of these glycan structures from a healthy, physiological state towards the diseased state. An AUC value over 0.5, and approaching 1, suggests both an increase in the accuracy and reliability of the test and an increasing trend in the relative intensities of the structures from a healthy to the diseased state. An opposite trend is observed when the AUC values are below 0.5 and approaching zero. For example, the AUC values for the glycan structures corresponding to m/z values 2676 and 2792 were 0.05±0.04 and 0.05±0.03, respectively. A decrease in the relative intensity of these two structures was observed in all cancer samples relative to the healthy ones. The ROC test AUC values were calculated here for all different glycan structures, as summarized in Table 3.

The changes in relative intensities were also evaluated using a single-factor ANOVA test. In this case, a statistically significant difference in the relative intensity of a glycan structure between cancer and healthy samples has been associated with a low p-value. Generally, a change associated with a p-value lower than 0.05 is considered to be significant, suggesting a 5% probability that the difference is due to chance. A list of the ANOVA test p-values for all glycan structures was compiled, as summarized in Table 3.

TABLE 3
Relative intensities of all glycans determined in this study and
their ROC analysis AUC values and ANOVA test p-derived from
blood sera of healthy individuals (n = 10) and prostate
cancer patients (n = 24).

| N- | | | | |
|---|---|---|---|---|
| glycan | Relative Intensity | | ROC analysis | |
| m/z | Healthy | Prostate Cancer | AUC ± | ANOVA test |
| value | Individuals | Patients | error | p-value |
| 1579.8 | 5.61 ± 0.25 | 4.54 ± 0.25 | 0.26 ± 0.09 | 0.019 |
| 1620.8 | 0.28 ± 0.01 | 0.38 ± 0.02 | 0.84 ± 0.07 | 0.014 |
| 1661.7 | 0.35 ± 0.11 | 0.19 ± 0.01 | 0.44 ± 0.14 | 0.053 |
| 1783.9 | 6.71 ± 0.23 | 5.18 ± 0.28 | 0.17 ± 0.07 | 0.003 |
| 1835.8 | 0.55 ± 0.02 | 0.90 ± 0.04 | 0.98 ± 0.02 | 4.3E−06 |
| 1865.9 | 0.48 ± 0.03 | 0.58 ± 0.02 | 0.72 ± 0.11 | 0.026 |
| 1906.9 | 1.04 ± 0.12 | 0.73 ± 0.04 | 0.21 ± 0.08 | 0.004 |
| 1989 | 1.46 ± 0.05 | 1.10 ± 0.10 | 0.20 ± 0.08 | 0.020 |
| 2040 | 0.70 ± 0.03 | 0.80 ± 0.04 | 0.70 ± 0.09 | 0.113 |
| 2070 | 0.53 ± 0.02 | 2.16 ± 0.20 | 1 | 1.9E−05 |
| 2081.1 | 0.13 ± 0.01 | 0.14 ± 0.01 | 0.73 ± 0.11 | 0.358 |
| 2111.1 | 0.77 ± 0.08 | 0.57 ± 0.03 | 0.30 ± 0.09 | 0.013 |
| 2186.1 | 0.90 ± 0.02 | 0.87 ± 0.04 | 0.38 ± 0.10 | 0.745 |
| 2192.1 | 1.92 ± 0.06 | 1.58 ± 0.12 | 0.24 ± 0.08 | 0.089 |
| 2244.1 | 0.66 ± 0.03 | 0.82 ± 0.04 | 0.78 ± 0.08 | 0.019 |
| 2285.2 | 0.24 ± 0.01 | 0.35 ± 0.01 | 0.94 ± 0.04 | 4.3E−05 |
| 2390.2 | 2.05 ± 0.23 | 2.26 ± 0.13 | 0.62 ± 0.12 | 0.424 |
| 2396.2 | 1.25 ± 0.07 | 1.05 ± 0.05 | 0.28 ± 0.10 | 0.031 |
| 2401.2 | 1.48 ± 0.16 | 1.39 ± 0.08 | 0.43 ± 0.13 | 0.601 |
| 2431.2 | 12.94 ± 0.20 | 13.75 ± 0.24 | 0.75 ± 0.09 | 0.059 |
| 2472.2 | 0.64 ± 0.02 | 0.55 ± 0.03 | 0.34 ± 0.10 | 0.076 |
| 2489.3 | 0.41 ± 0.02 | 0.50 ± 0.02 | 0.80 ± 0.09 | 0.010 |
| 2605.3 | 2.78 ± 0.06 | 2.89 ± 0.09 | 0.55 ± 0.10 | 0.491 |
| 2646.3 | 0.39 ± 0.02 | 0.38 ± 0.02 | 0.41 ± 0.10 | 0.794 |
| 2676.3 | 1.25 ± 0.03 | 0.96 ± 0.04 | 0.05 ± 0.04 | 3.3E−05 |
| 2792.4 | 25.22 ± 1.27 | 18.48 ± 0.47 | 0.05 ± 0.03 | 1.1E−06 |
| 2809.4 | 1.82 ± 0.73 | 1.06 ± 0.19 | 0.43 ± 0.14 | 0.195 |
| 2850.4 | 1.10 ± 0.04 | 1.22 ± 0.06 | 0.63 ± 0.10 | 0.196 |
| 2880.4 | 1.11 ± 0.07 | 1.38 ± 0.06 | 0.77 ± 0.09 | 0.015 |
| 2925.4 | 0.48 ± 0.03 | 0.54 ± 0.03 | 0.58 ± 0.10 | 0.335 |
| 2966.5 | 4.87 ± 0.16 | 5.02 ± 0.14 | 0.53 ± 0.11 | 0.568 |
| 3037.3 | 0.26 ± 0.01 | 0.26 ± 0.01 | 0.55 ± 0.12 | 0.873 |
| 3054.5 | 0.40 ± 0.02 | 0.76 ± 0.04 | 0.97 ± 0.03 | 5.9E−06 |
| 3211.6 | 1.27 ± 0.06 | 1.14 ± 0.06 | 0.69 ± 0.10 | 0.182 |
| 3241.6 | 1.54 ± 0.07 | 3.84 ± 0.23 | 1 | 7.2E−07 |
| 3385.6 | 0.15 ± 0.01 | 0.19 ± 0.02 | 0.76 ± 0.09 | 0.240 |
| 3415.7 | 0.62 ± 0.03 | 2.34 ± 0.17 | 1 | 2.9E−07 |
| 3602.8 | 7.32 ± 0.57 | 7.80 ± 0.35 | 0.58 ± 0.12 | 0.481 |
| 3690.8 | 0.52 ± 0.03 | 0.88 ± 0.04 | 0.95 ± 0.03 | 4.1E−06 |
| 3776.9 | 5.34 ± 0.38 | 6.05 ± 0.46 | 0.56 ± 0.11 | 0.363 |
| 3864.9 | 0.19 ± 0.02 | 0.41 ± 0.03 | 0.95 ± 0.04 | 5.8E−04 |
| 3951 | 0.33 ± 0.02 | 0.42 ± 0.05 | 0.63 ± 0.10 | 0.301 |
| 3963.9 | ND | ND | — | — |
| 4052 | 0.57 ± 0.05 | 0.99 ± 0.06 | 0.89 ± 0.05 | 3.4E−04 |
| 4226.1 | 0.33 ± 0.03 | 0.66 ± 0.05 | 0.94 ± 0.04 | 9.3E−04 |
| 4413.2 | 0.79 ± 0.09 | 0.79 ± 0.07 | 0.48 ± 0.12 | 0.999 |
| 4587.3 | 0.51 ± 0.06 | 0.61 ± 0.06 | 0.46 ± 0.11 | 0.322 |
| 4761.4 | 0.22 ± 0.01 | 0.29 ± 0.06 | 0.61 ± 0.12 | 0.556 |
| 4862.4 | ND | 0.07 ± 0.01 | — | — |

ND—not detected

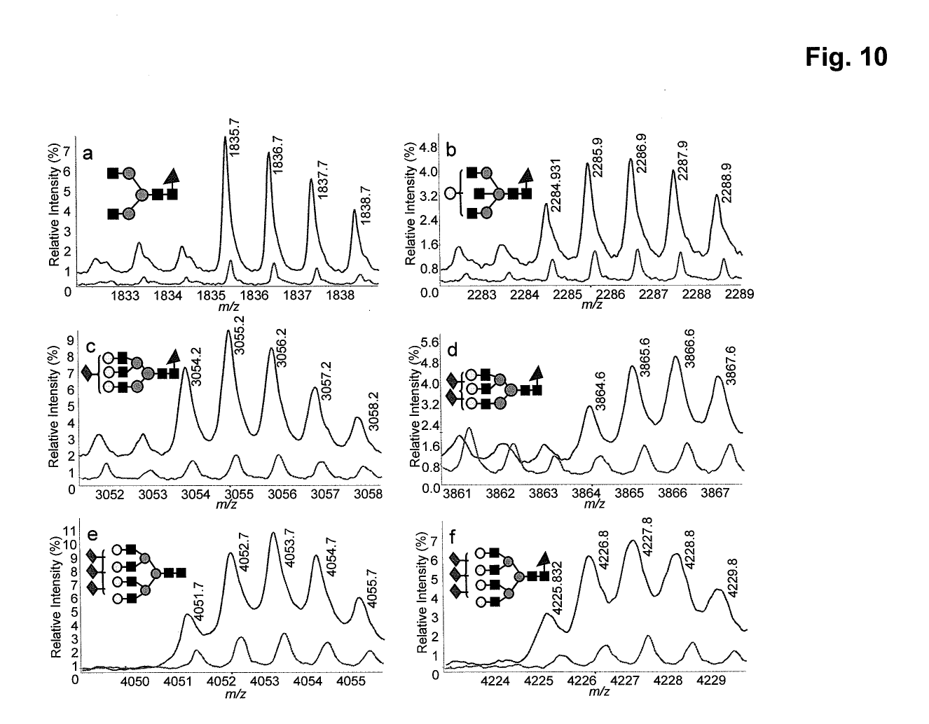

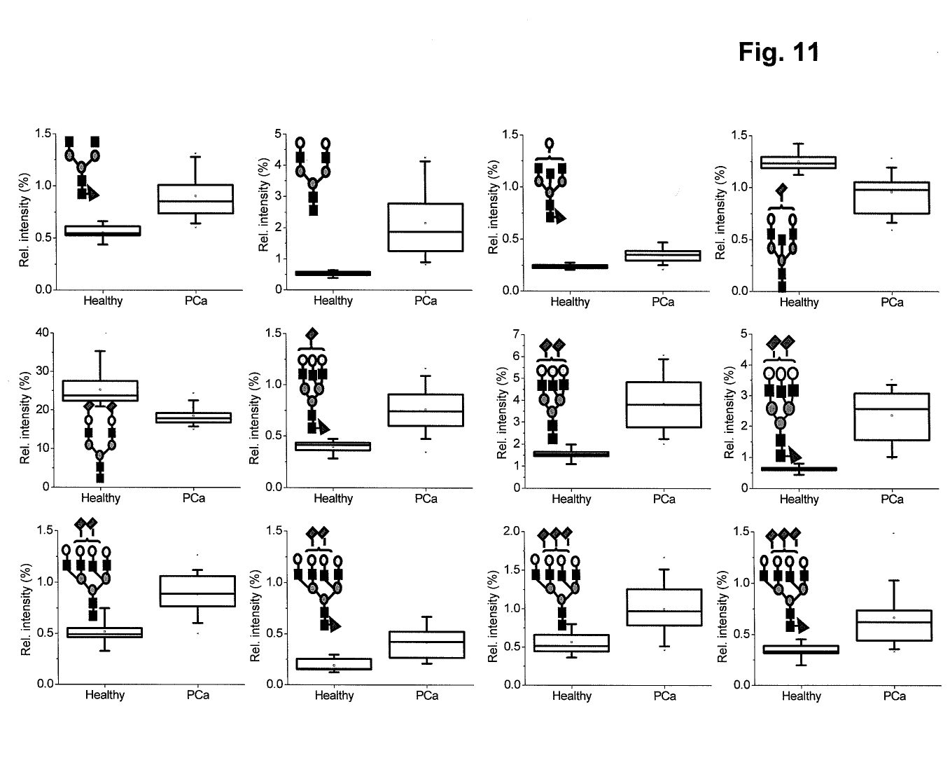

The AUC values and their corresponding ANOVA test p-values for the glycan structures with a moderate to highly accurate ROC analysis are listed in Table 4. Twelve of the listed N-glycans, which are highlighted in Table 4, have both ROC analysis AUC values reflecting high accuracy and p-values lower than 0.001. Spectra for six of the twelve structures reflecting the changes in the relative intensities between healthy and cancer samples are depicted in FIG. 10, while a bar graph illustrating the overall differences for all samples is shown in FIG. 11. Two of the twelve structures demonstrated a decrease in their relative intensity for all prostate cancer samples, while the other 10 demonstrated an increase as a result of cancer progression. Moreover, nine of the twelve N-glycans were sialylated to a different degree (mono-, di-, and trisialylated structures), while six were fucosylated.

TABLE 4
Area under the curve (AUC) values from ROC analysis and p-values from
ANOVA test for N-glycans derived from human blood serum of healthy individuals and prostate
cancer patients. Highlighted rows are the N-glycans which received a highly accurate ROC
analysis AUC values (0.9 < AUC < 1 or 0 < AUC < 0.1) and a low ANOVA test p-values, while
others are N-glycans which received a moderately accurate ROC analysis AUC values
(0.7 < AUC < 0.9 or 0.1 < AUC < 0.3) and higher ANOVA p-test values.

| | | | ANOVA |
|---|---|---|---|
| N-glycan | Structure | AUC ± error | p-value |
| 1579.8 | 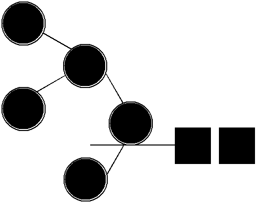 | 0.26 ± 0.09 | 0.019 |
| 1620.8 | 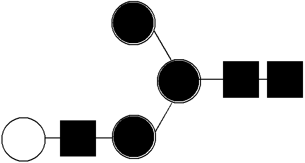 | 0.84 ± 0.07 | 0.014 |
| 1783.9 | 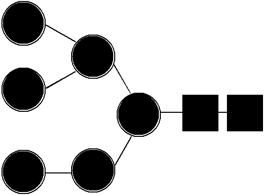 | 0.17 ± 0.07 | 0.003 |
| 1835.8 | 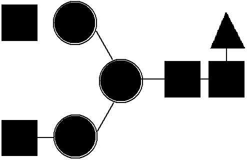 | 0.98 ± 0.02 | 4.3E−06 |
| 1865.9 | 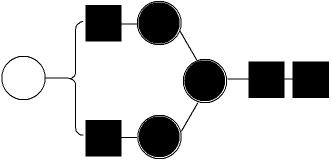 | 0.72 ± 0.10 | 0.026 |
| 1906.9 | 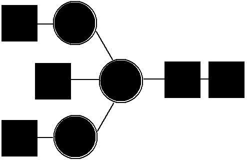 | 0.21 ± 0.08 | 0.004 |
| 1989.1 | 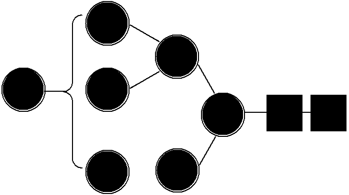 | 0.20 ± 0.08 | 0.020 |
| 2070 | 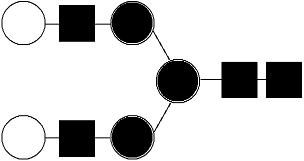 | 1 | 1.9E−05 |
| 2081.1 | 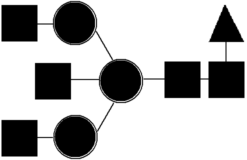 | 0.73 ± 0.11 | 0.358 |
| 2111.1 | 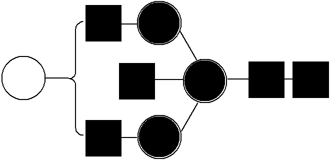 | 0.30 ± 0.09 | 0.013 |
| 2192.1 | 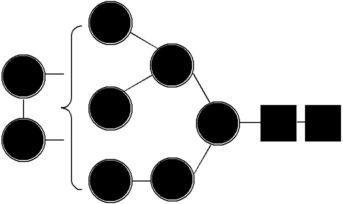 | 0.24 ± 0.08 | 0.089 |
| 2244.1 | 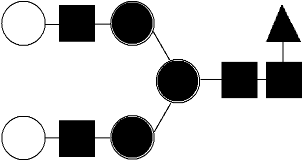 | 0.78 ± 0.08 | 0.019 |
| 2285.2 | 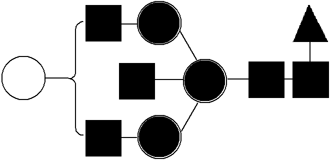 | 0.94 ± 0.04 | 4.3E−05 |
| 2396.2 | 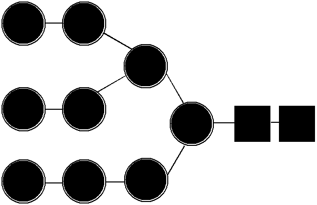 | 0.28 ± 0.10 | 0.031 |
| 2431.2 | 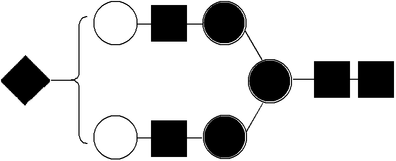 | 0.75 ± 0.09 | 0.059 |
| 2489.3 | 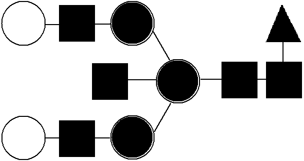 | 0.80 ± 0.09 | 0.010 |
| 2676.3 | 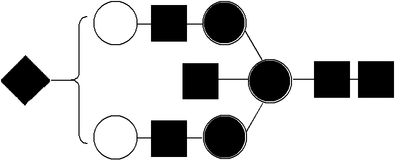 | 0.05 ± 0.04 | 3.0E−05 |
| 2792.4 | 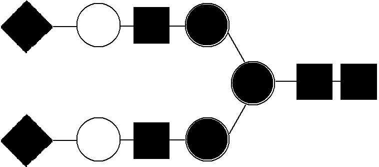 | 0.05 ± 0.03 | 1.1E−06 |
| 2880.4 | 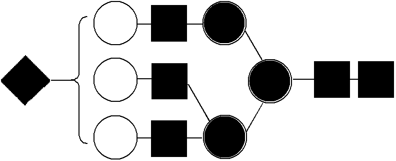 | 0.77 ± 0.09 | 0.015 |
| 3054.5 | 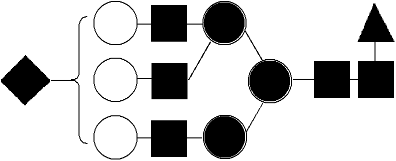 | 0.97 ± 0.03 | 5.9E−0.6 |
| 3241.6 | 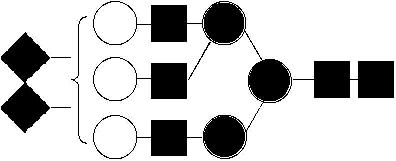 | 1 | 7.2E−07 |
| 3385.6 | 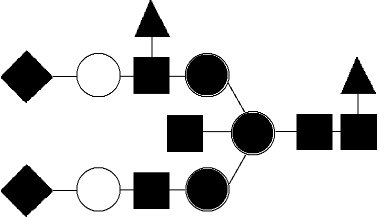 | 0.76 ± 0.09 | 0.239 |
| 3415.7 | 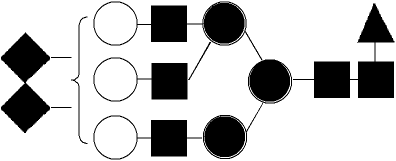 | 1 | 2.9E−0.7 |
| 3690.8 | 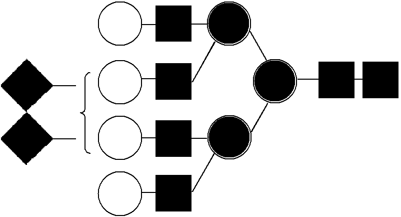 | 0.95 ± 0.03 | 4.1E−06 |
| 3864.9 | 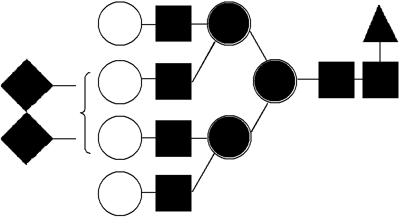 | 0.95 ± 0.04 | 5.8E−04 |
| 4052.0 | 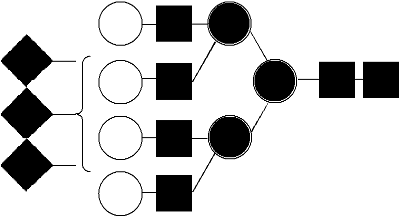 | 0.89 ± 0.05 | 3.4E−04 |
| 4226.1 | 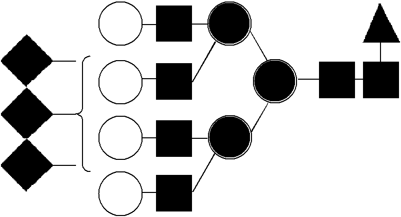 | 0.94 ± 0.04 | 9.3E−0.4 |

Thus, the utility of selected N-glycan structures as potential tumor biomarkers for identifying the presence of prostate cancer is demonstrated through their significant relative differences for twelve glycan structures in the samples from the metastatic prostate patients. Moreover, these findings were confirmed by two independent statistical approaches which are validated well in biomedical research.

Discussion. The analytical approach discussed here utilized a 10-μl aliquot of unfractionated human serum to generate glycomic profiles through solid-phase permethylation and MALDI-MS. Although the demonstrated data provide insight into the mechanisms of altered glycosylation and point to the structural changes of glycoproteins at the onset and during the course of cancer development, no information pertaining to the proteins which endured such glycosylation changes was gathered. Nevertheless, the potential of employing the glycomic approach as a diagnostic and prognostic tool has been demonstrated.

The differences in relative intensities of over 50 N-glycans were measured and relative intensity changes appeared in accordance with the presence of disease. As demonstrated in this study, the glycomic profiles feature a significant increase in total fucosylation in correlation with presence of malignancy. Six of the twelve glycan structures, demonstrating a significant increase from healthy to prostate cancer state in this study, were fucosylated. This correlates with a recent report that alpha1, 2-L-fucosyltranferase exhibits an increased activity in prostate carcinoma LNPaC cells. Different types of cancer, such as pancreatic cancer, colorectal cancer, human leucocyte cancer, hepatocarcinomas, and renal carcinomas also show a malignancy associated with increased fucosylation.

An additional analytical asset of our procedure is that both sialylated and non-sialylated structures can be concurrently analyzed due to the use of solid-phase permethylation, thus permitting structural correlations between neutral and sialylated glycan structures. Thus far, no significant sialylation alterations for N-glycans were observed in this study. O-glycans were not investigated. The principal component analysis of the two groups of samples analyzed in this study demonstrates a distinct clustering with a significant difference between the two groups. In fact, it has resulted in a complete separation between the two groups of data, demonstrating the utility of using glycomic profiling from serum in studies investigating the effects of different therapies, a close follow-up of disease conditions, etc.

The diagnostic potential of MS-based glycomic profiles has been demonstrated. A 10-μl volume of human serum is sufficient for typical analyses. Differential profiles from healthy individuals and prostate cancer patients were adequately tested using statistical tools such as PCA, ROC and ANOVA. All statistical analyses confirmed differences in the N-glycosylation patterns of healthy subjects vs. prostate cancer patients. The bioanalytical information acquired here provides some insight into the mechanisms of aberrant glycosylation and point to the structural changes of glycoproteins. These methodologies are useful for new pre-screening methods to aid early prostate cancer detection through the determination of glycan-specific biomarkers. Such highly indicative biomarker candidates may also identify patients destined to have a short versus a long remission from ADT.

### Liver Cancer and Cirrhosis

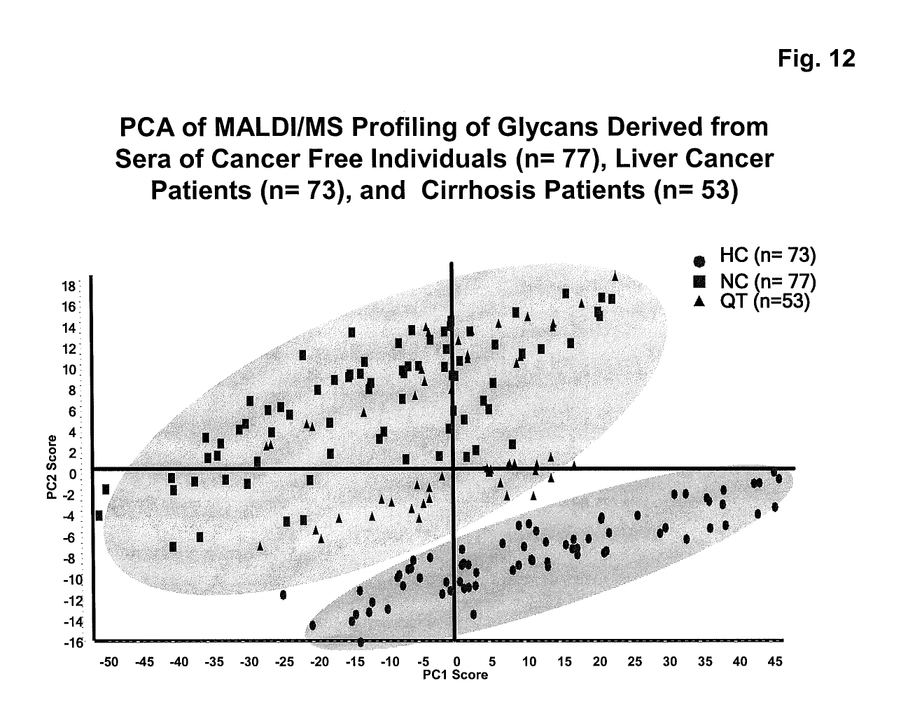

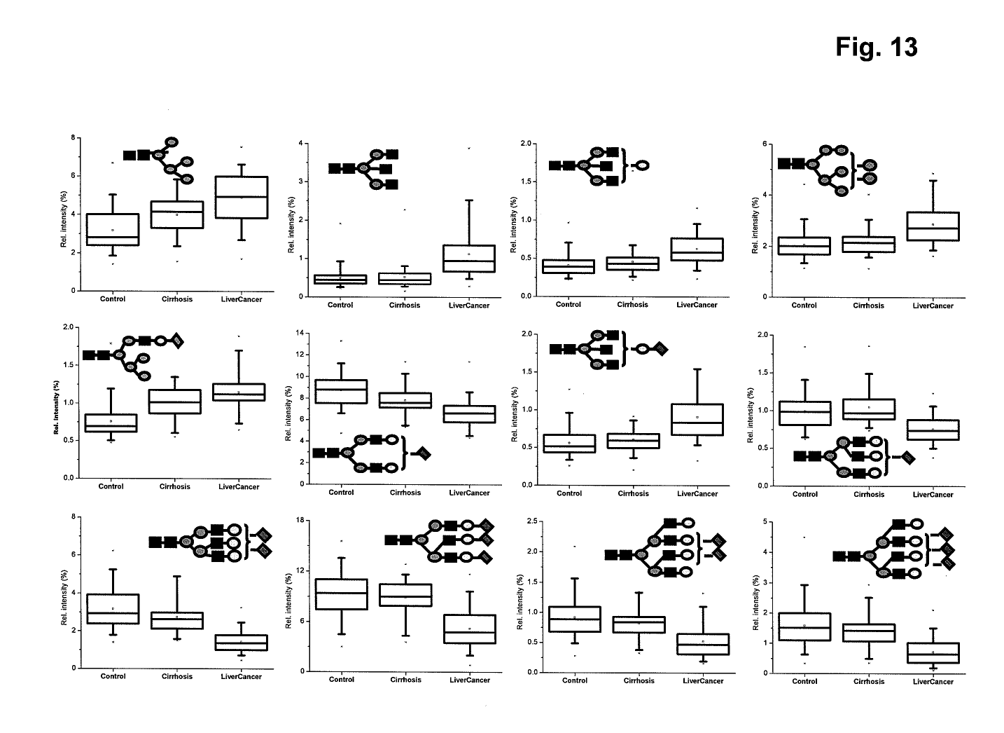

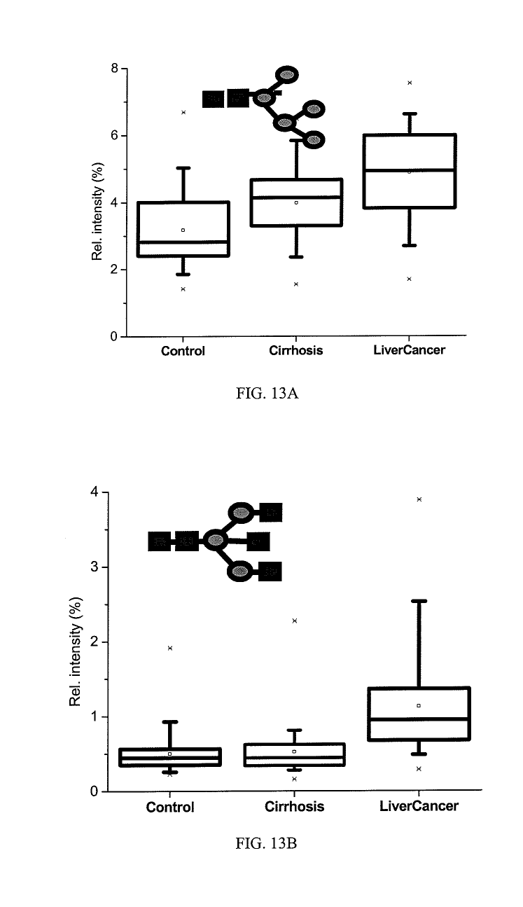

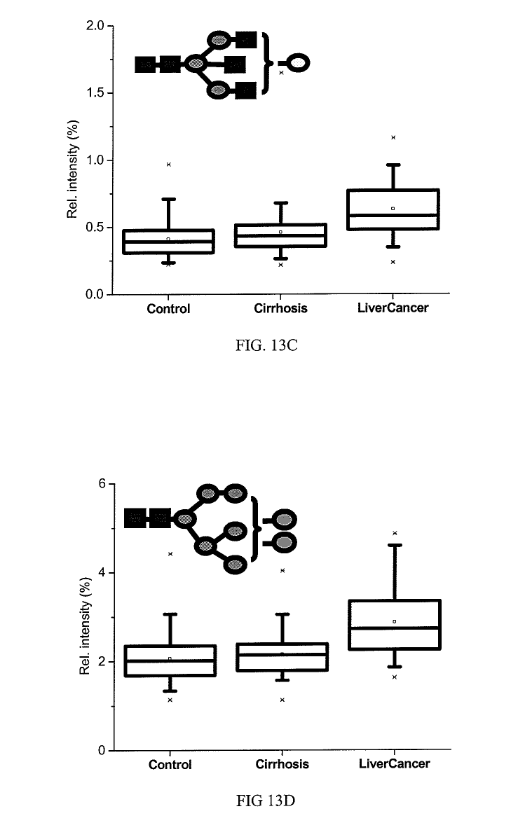

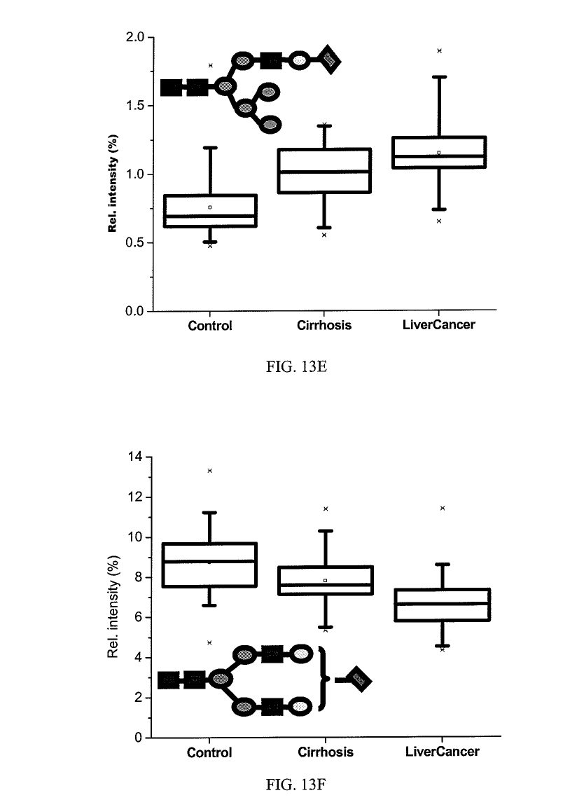

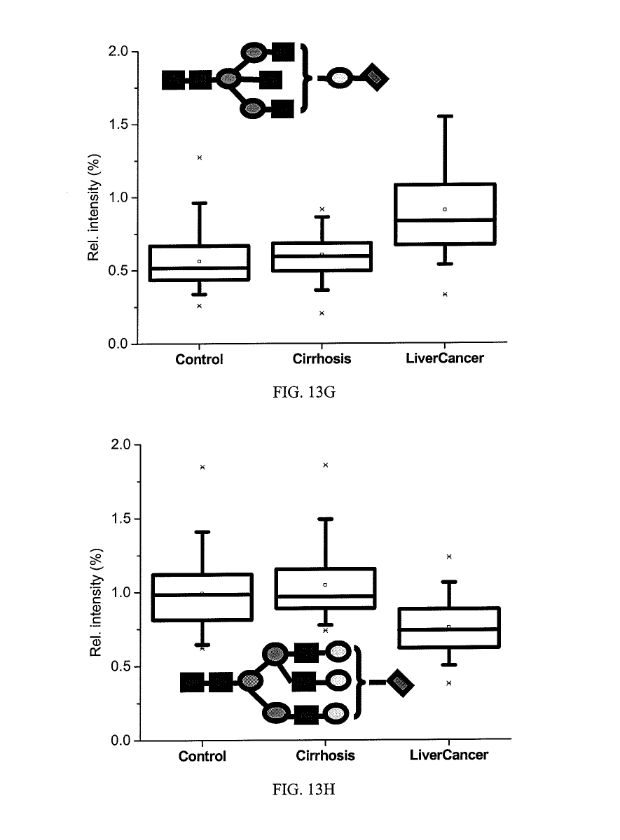

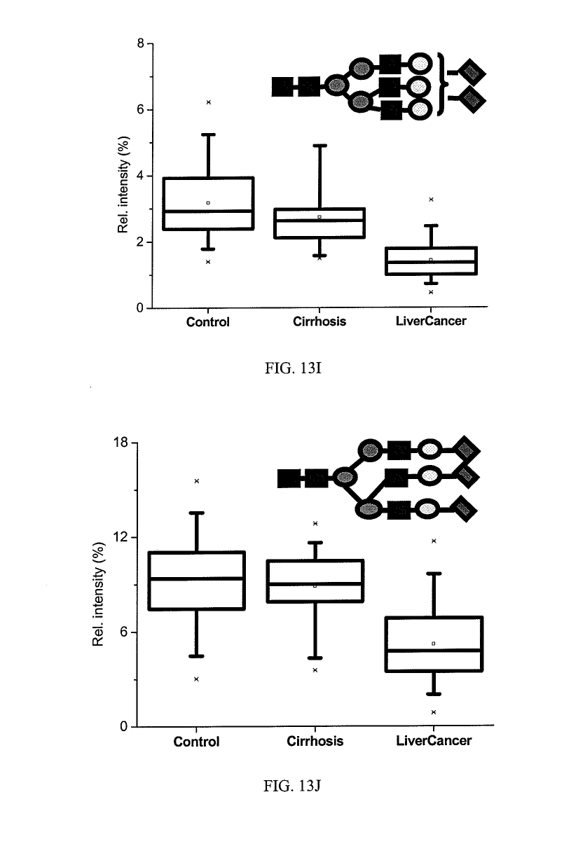

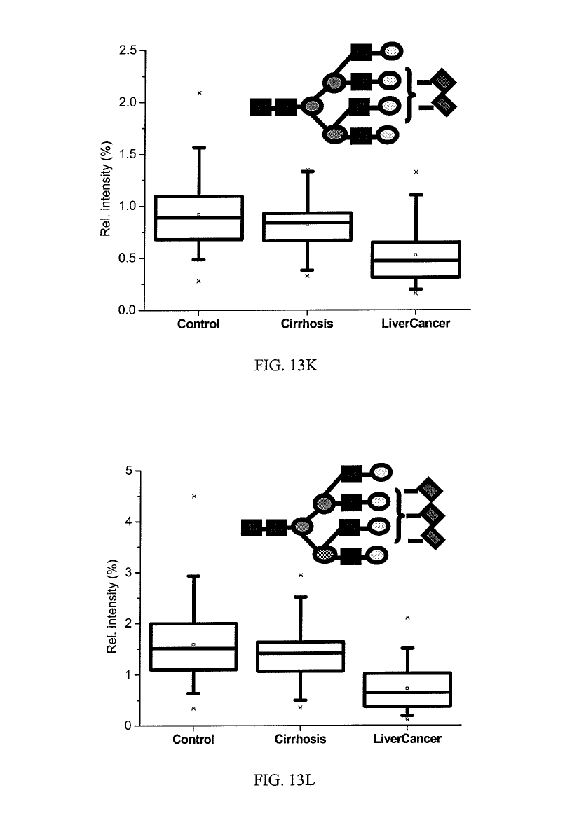

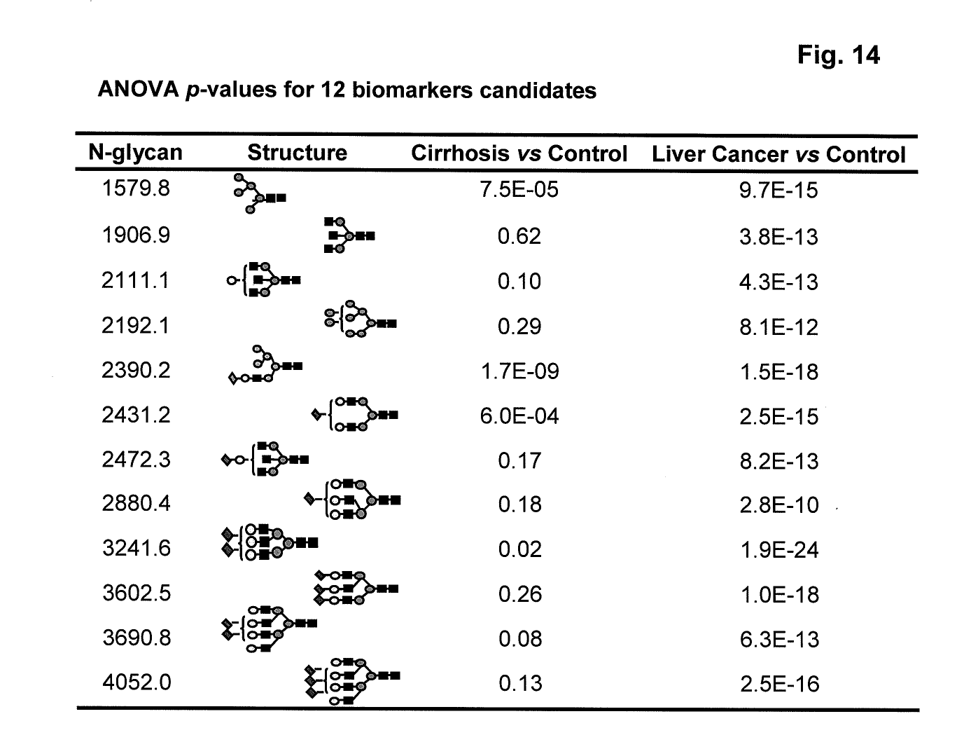

PCA score plots for MALDI/MS of N-glycans derived from the sera of cancer-free individuals, liver cancer patients, and cirrhosis patients; and the ROC and ANOVA analyses therefor are shown in FIGS. 12-14.

### Materials and Methods

Materials. The endoglycosidase, Peptide-N-Glycosidase F (PNGase F; EC 3.5.1.52), isolated from E. coli, used for deglycosylation, was obtained from Cape Cod Company (East Falmouth, Mass.). Trypsin (E.C.: 3.4.21.4) was obtained from Sigma (St. Louis, Mo.). Trifluoroethanol (TFE), 2,5-dihydroxybenzoic acid (DHB), sodium hydroxide, 3-[3-(cholamidopropyl)dimethylammonio]-1-propanesulfonate (CHAPS), Tris-HCl, sodium pyrophosphate, ethylenediamine tetraacetic acid (EDTA) and ethylene glycol bis(2-aminoethyl ether)tetraacetic acid (EGTA) were purchased from Aldrich (Milwaukee, Wis.). Chloroform, iodomethane and sodium chloride were received from EM Science (Gibbstown, N.J.). Dithiothreitol (DTT) and iodoacetamide (IAA) were the products of Bio-Rad Laboratories (Hercules, Calif.). Ammonium bicarbonate was received from Mallinckrodt Chemical Company (Paris, Ky.). Acetonitrile (ACN) was purchased from Fisher Scientific (Fair Lawn, N.J.). All other common chemicals of analytical-grade quality were purchased from Sigma (St. Louis, Mo.).

Serum samples and clinical diagnosis—Breast Cancer Patients. Blood serum collections from volunteer healthy individuals and the patients diagnosed with different breast cancer stages were performed by a clinical team. All serum samples were collected from females. Venous blood samples were taken during the morning fasting state, with minimal stasis in evacuated tubes. After at least 30 min, but within 2 h, the tubes were centrifuged at 20° C. for 12 min at 1200 g. Sera were stored frozen in plastic vials at −80° C. before use in consecutive measurements.

The glycomic profiles were generated for samples derived from women in one of the following categories: (1) disease-free woman at low risk for developing breast cancer; (2) post-menopausal women with a confirmed disease stratified according to their having non-invasive or invasive breast cancer. These were distributed into 4 subgroups (I-IV) according to the severity of breast cancer development. The clinical study was approved by the local ethical committees of Indiana University in Bloomington and Indianapolis, and the Greater Baltimore Medical Center. Informed consent was obtained from all serum donors.

Serum samples and clinical diagnosis—Prostate Cancer Patients. Serum from clotted human male whole blood samples (healthy individuals) and serum samples from patients with documented metastatic prostate cancer were collected using an IUPUI IRB approved clinical trial conducted through the Hoosier Oncology Group. Venous blood samples were taken in the morning's fasting state, being collected with the minimal stasis in evacuated tubes. After at least 30 min, but within 2 h, the tubes were centrifuged at 20° C. for 12 min at 1200 g, and the sera were stored frozen in plastic vials at −80° C. until the time of the consecutive analyses.

The glycomic profiles were generated for the samples derived from men in the following two categories: (1) non-diseased healthy individuals; and (2) men with confirmed prostate cancer who were undergoing androgen-deprivation therapy (ADT). In this study, the serum for the latter was obtained at the time of starting ADT. As such, the patients enrolled on this study had a documented disease burden of prostate cancer.

Cancer cell lines. Cancer cell lines (Normal mammary epithelial: MCF10A invasive: MDA-MB-231, MDA-MB-435, non-invasive: 578T, ADR-RES, BT549 and T47D) were lysed in a CHAPS-based buffer (150 mM NaCl, 0.5% CHAPS, 50 mM Tris (pH 7.5), 10 mM sodium pyrophosphate, 1 mM EDTA, 1 mM EGTA). Lysates were then subjected to ultracentrifugation at 40,000 rpm, separating the samples into cytosolic and membrane-associated proteins. Protein aliquots (200-μg, determined by Bradford assay) were subjected to the same procedures described for the blood serum.

Reduction, alkylation and release of N-glycans from glycoproteins. Human serum samples were reduced and alkylated as previously described. Briefly, a 10-μl aliquot of human blood serum was lyophilized and then resuspended in 25 μl of 25 mM ammonium bicarbonate, 25 μl of TFE and 2.5 μl of 200 mM DTT prior to incubation at 60° C. for 45 min. A 10-μl aliquot of 200 mM IAA was then added and allowed to react at room temperature for 1 h in the dark. Subsequently, a 2.5-μl aliquot of DTT was added to react with the excess IAA. Next, the reaction mixture was diluted with 300 μl of water and 100 μl of ammonium bicarbonate stock solution to adjust pH to 7.5- 8.0.

For reduced/alkylated samples from breast cancer patients, proteolytic digestion was performed with 1 μg/μl [or 1:50 (w/w) ratio] proteomics-grade trypsin dissolved in 1 mM HCl and incubated at 37° C. overnight (at least 18 h). Afterwards, enzyme was quenched by incubation at 95° C. for 10 min and allowed to cool at room temperature. The N-glycans were enzymatically released from human serum samples as previously described. Briefly, a 5 mU of PNGase F was added to the reaction mixture, which was subsequently incubated overnight (18-22 h) at 37° C.

For reduced/alkylated samples from prostate cancer patients, proteolytic digestion was not performed with trypsin. Rather, the N-glycans were enzymatically released from human serum samples as previously described. Briefly, a 5 mU of PNGase F was added to the reaction mixture, which was subsequently incubated overnight (18-22 h) at 37° C.

Solid-phase extraction of enzymatically released N-glycans. The volume of enzymatically released glycans was adjusted to 1 ml by adding deionized water. Samples were then applied to both C18 Sep-Pak® cartridges (Waters, Milford, Mass.) and activated charcoal cartridges (Harvard Apparatus, Holliston, Mass.). The use of C18 Sep-Pak® cartridges is for isolation of the glycans from peptides and proteins, which would otherwise interfere with trapping on the activated charcoal cartridges. The reaction mixture was first applied to C18 Sep-Pak® cartridge that had been preconditioned with ethanol and deionized water according to the manufacturer's recommendation. The reaction mixture was circulated through the C18 Sep-Pak® cartridge 5 times prior to washing with water. Peptides and O-linked glycopeptides were retained on the C18 Sep-Pak® cartridge, while the released glycans were collected as eluents. Next, the C18 Sep-Pak® cartridge was washed with 1 ml of deionized water. The combined eluents containing the released N-glycans were then passed over activated charcoal microcolumns. The columns were pre-conditioned with 1 ml of ACN and 1 ml of 0.1% trifluoroacetic acid (TFA) aqueous solution, as recommended by the manufacturer. After applying the sample, the microcolumn was washed with 1 ml of 0.1% TFA aqueous solution. The samples were then eluted with a 1-ml aliquot of 50% ACN aqueous solution containing 0.1% TFA. Finally, the purified glycans were evaporated to dryness using vacuum CentriVap Concentrator (Labconco Corporation, Kansas City, Mo.) prior to solid-phase permethylation.

Solid-phase permethylation. Permethylation of enzymatically released and solid-phase purified N-glycans was accomplished utilizing our recently published solid-phase permethylation technique. This approach involves packing of sodium hydroxide beads in peek tubes (1 mm i.d.; Polymicro Technologies, Phoenix, Ariz.), permitting complete derivatization. Tubes, nuts and ferrules from Upchurch Scientific (Oak Harbor, Wash.) were employed for assembling the sodium hydroxide capillary reactor. Sodium hydroxide powder was suspended in ACN for packing. A 100-μl syringe from Hamilton (Reno, Nev.) and a syringe pump from KD Scientific, Inc. (Holliston, Mass.) were employed for introducing the sample into the reactor. Sodium hydroxide reactor was first conditioned with 60 μl of dimethyl sulfoxide (DMSO) at a 5 μl/min flow rate. Samples were resuspended in DMSO and mixed with methyl iodide solution containing traces of deionized water. Typically, released and purified N-glycans were resuspended in a 50-μl aliquot of DMSO, to which 0.3 μl of water and 22 μl methyl iodide were added. This permethylation procedure has been shown to minimize oxidative degradation, peeling reactions as well as to avoid the need of excessive clean-up. Sample was infused through the reactor at a slow flow rate of 2 μl/min. The reactor was then washed with 230 μl ACN (flow rate: 5 μl/min). All eluents were combined, and the permethylated N-glycans were finally extracted using 200 μl chloroform and washed repeatedly (3 times) with 200 μl of water prior to drying.

MALDI-TOF MS instrumentation. Permethylated glycans were resuspended in 2 μl of (50:50) methanol:water solution. A 0.5-μl aliquot of the sample was then spotted directly on the MALDI plate and mixed with the equal volume of DHB-matrix prepared by suspending 10 mg of DHB in 1 ml of (50:50) water:methanol solution, containing 1 mM sodium acetate. The inclusion of sodium acetate is to promote a nearly complete sodium adduct formation in MALDI-MS. The MALDI plate was then dried under vacuum to ensure uniform crystallization. Mass spectra were acquired using the Applied Biosystems 4800 MALDI TOF/TOF Analyzer (Applied Biosystems Inc., Framingham, Mass.). This instrument is equipped with Nd:YAG laser with 355-nm wavelength. MALDI-spectra were recorded solely in the positive-ion mode, since permethylation eliminates the negative charge normally associated with sialylated glycans. Argon gas was used as a collision gas in the tandem MS measurements, and the collision cell pressure was set to 6.5×10⁻⁶ torr. The acquired spectra were the average of 1000 laser-shots.

Data evaluation. The obtained MALDI-MS data were further processed using DataExplorer 4.0 (Applied Biosystems, Framingham, Mass.) to generate ASCII files listing m/z values and intensities. An in-house developed software tool (PeakCalc 2.0) was then used to extract the intensities of N-glycans. Principal component analysis (PCA) was performed using MarkerView (ABI, Framingham, Mass.), allowing the visualization of multivariate information. Supervised PCA methods were employed, using a prior knowledge of the sample groups as healthy vs. diseased. MS data were weighted using the base-e logarithm of the peak intensities. The peak intensities were also scaled using pareto option in which each value is subtracted by the average and divided by the square root of the standard deviation. This option is suitable for MS data, since it prevents intense peaks from completely dominating the PCA process, thus allowing any peak with good signal-to-noise ratio to contribute.

We also used Receiver Operating Characteristics (ROC) curve analysis using AccuROC 2.5 software for Windows (Accumetric Corporation, Montreal, Canada) to assess the sensitivity and selectivity of the potential diagnostic variables. ROC curve is defined as a plot of test sensitivity, in its y-axis versus its specificity or false positive rate as the x-axis. This type of statistical analysis is an effective method of evaluating the quality or performance of diagnostic tests, and has been widely used in radiology to evaluate performance of many radiological tests.

Our data were also statistically analyzed using a single factor Analysis of Variance (ANOVA) test. A difference between the two groups of data was considered statistically significant when p-values were less then 0.05, suggesting a less than 5% probability that the difference between the two groups is statistically not significant.

The range of values throughout this study was expressed as a standard error of mean (SEM) value, which accounts for a sample size. Standard deviation is the most common measure of statistical dispersion, measuring how spread-out the values in a data set appear (e.g., due to limitations in measurement reproducibility). However, when working with biological samples, any observed variation observed might be intrinsic to the phenomenon that distinct members of a population differ greatly (biochemical individuality). Consequently, the standard error (SE), or SEM, signifies an estimate of the standard deviation of the sampling distribution of means, based on the data from one or more random samples. SEM then accounts for the number of real samples, implicating their biodiversity in the evaluation process.

While the invention has been illustrated and described in detail in the foregoing description, such an illustration and description is to be considered as exemplary and not restrictive in character, it being understood that only the illustrative embodiments have been described and that all changes and modifications that come within the spirit of the invention are desired to be protected. Those of ordinary skill in the art may readily devise their own implementations that incorporate one or more of the features described herein, and thus fall within the spirit and scope of the present invention.

We claim:

1. A method for monitoring glycomic change in a biological system, comprising:
establishing a glycomic profile for said biological system in a base state,
establishing a glycomic profile for said biological system in a perturbed state, and
comparing said glycomic profiles to identify a glycomic change occurring in said perturbed state.

2. The method of claim 1, wherein the biological system is that of a human being.

3. The method of claim 2, wherein the base state is associated with the absence of a disease.

4. The method of claim 3, wherein the perturbed state is associated with the presence of a disease.

5. The method of claim 4, wherein the disease is selected from the group consisting of breast cancer, prostate cancer, liver cancer, and cirrhosis.

6. The method of claim 5, wherein the glycomic profiles display one or more permethylated N-glycan.

7. The method of claim 6, wherein the glycomic change is represented by a statistically significant increase or decrease in at least one permethylated N-glycan.

8. The method of claim 7, wherein the at least one permethylated N-glycan is a disease biomarker.

9. The method of claim 8, wherein the glycomic profiles are established by a method comprising:
conducting solid-phase permethylation of N-glycans from human serum to afford permethylated N-glycans, and
analyzing said permethylated N-glycans by MALDI-MS mass spectrometry.

10. The method of claim 9, further comprising the step of removing peptides and O-glycopeptides by RPLC prior to conducting solid-phase permethylation of N-glycans.

11. The method of claim 10, wherein solid-phase permethylation of N-glyeans is conducted by a method comprising:
infusing a polar, aprotic solvent through a packed inorganic base, wherein the solvent includes an N-glycan and a source of methyl groups;
contacting the N-glycan with the source of methyl groups; and
collecting a permethylated N-glycan.

12. The method of claim 11, wherein the base is NaOH.

13. The method of claim 11, wherein the solvent is DMSO.

14. The method of claim 11, wherein the source of methyl groups is methyl iodide.

15. A permethylated N-glycan biomarker for breast cancer identified by the method of claim 8.

16. The N-glycan biomarker of claim 15, wherein the N-glycan is sialylated and/or fucosylated.

17. A permethylated N-glycan biomarker for prostate cancer identified by the method of claim 8.

18. The N-glycan biomarker of claim 17, wherein the N-glycan is sialylated and/or fucosylated.

19. A permethylated N-glycan biomarker for liver cancer identified by the method of claim 8.

20. A permethylated N-glycan biomarker for cirrhosis identified by the method of claim 8.
